(19) United States
(12) Patent Application Publication
(10) Pub. No.: US 2022/0151567 A1
(43) Pub. Date: May 19, 2022

(54) JOINT ASSESSMENT OF MYOCARDIAL STRAIN AND INTRACARDIAC BLOOD FLOW

(71) Applicant: Siemens Healthcare GmbH, Erlangen (DE)

(72) Inventors: Teodora Chitiboi, Jersey City, NJ (US); Viorel Mihalef, North Brunswick, NJ (US); Puneet Sharma, Princeton Junction, NJ (US); Tiziano Passerini, Plainsboro, NJ (US)

(21) Appl. No.: 16/950,046

(22) Filed: Nov. 17, 2020

(51) Int. Cl. A61B 5/00; A61B 5/029; A61B 5/055; G06N 20/00; G16H 30/20; G16H 50/50

(52) U.S. Cl. CPC A61B 5/7275; A61B 5/0044; A61B 5/029; A61B 5/7264; G16H 50/50; A61B 5/055; G06N 20/00; G16H 30/20; A61B 5/7267

(57) ABSTRACT

Systems and methods for determining myocardium strain and intracardiac blood flow data of the heart are provided. Input medical imaging data of a heart of a patient is received. At least one of extracted myocardium strain data of the heart and extracted intracardiac blood flow data of the heart is determined from the input medical imaging data. At least one of predicted myocardium strain data of the heart and predicted intracardiac blood flow data of the heart is determined based on the at least one of the extracted myocardium strain data and the extracted intracardiac blood flow data using a model of the heart. The model of the heart jointly models myocardium strain of the heart and intracardiac blood flow of the heart. The at least one of the predicted myocardium strain of the heart and the predicted intracardiac blood flow of the heart is output.

### TECHNICAL FIELD

The present invention relates generally to the assessment of cardiovascular diseases, and in particular to the joint assessment of myocardial strain and intracardiac blood flow for the assessment of cardiovascular diseases.

### BACKGROUND

Diagnostic imaging plays an important role in the assessment of cardiovascular diseases such as HCM (hypertrophic cardiomyopathy), congenital aortic stenosis, and valve diseases. One approach for the assessment of cardiovascular disease from diagnostic imaging is myocardial strain analysis, where strain patterns in myocardial tissue depicted in a time-varying MRI (magnetic resonance imaging) series are analyzed. Another approach for the assessment of cardiovascular disease from diagnostic imaging is intracardiac blood flow analysis, where blood flow within the heart depicted in a time-varying MRI is analyzed. Historically, myocardial strain analysis and intracardiac flow analysis were independently developed by different MRI research communities focusing exclusively on only one of the approaches. Accordingly, there are no existing systems for handling the joint analysis of myocardial strain and intracardiac blood flow.

### BRIEF SUMMARY OF THE INVENTION

In accordance with one or more embodiments, systems and methods for joint analysis of myocardium strain and intracardiac blood flow data of the heart are provided. Input medical imaging data of a heart of a patient is received. At least one of extracted myocardium strain data of the heart and extracted intracardiac blood flow data of the heart is determined from the input medical imaging data. At least one of predicted myocardium strain data of the heart and predicted intracardiac blood flow data of the heart is determined based on the at least one of the extracted myocardium strain data and the extracted intracardiac blood flow data using a model of the heart. The model of the heart jointly models myocardium strain of the heart and intracardiac blood flow of the heart. The at least one of the predicted myocardium strain of the heart and the predicted intracardiac blood flow of the heart is output.

In one embodiment, the model of the heart is a computational model of the heart. The computational model of the heart is updated based on the extracted myocardium strain data of the heart and extracted intracardiac blood flow data. Physiological function of the heart is simulated using the updated computational model to determine the at least one of the predicted myocardium strain data of the heart and the predicted intracardiac blood flow data of the heart.

In one embodiment, the model of the heart is a machine learning based model of the heart. The predicted myocardium strain data of the heart is predicted from the extracted intracardiac blood flow data of the heart using the machine learning based model and the predicted intracardiac blood flow data of the heart is predicted from the extracted myocardium strain data of the heart using the machine learning based model. In one embodiment, the machine learning based model of the heart comprises a first generator network for predicting the predicted myocardium strain data of the heart from the extracted intracardiac blood flow data of the heart and a second generator network for predicting the predicted intracardiac blood flow data of the heart from the extracted myocardium strain data of the heart. The first generator network and the second generator network are jointly trained through an adversarial training with a first discriminator network and a second discriminator network respectively.

In one embodiment, the at least one of the predicted myocardium strain of the heart and the predicted intracardiac blood flow of the heart is displayed overlaid on the input medical imaging data or the at least one of the predicted myocardium strain of the heart and the predicted intracardiac blood flow of the heart is displayed overlaid on a graphical representation of the heart of the patient generated based on the input medical imaging data.

In one embodiment, progression of a disease is simulated using the model of the heart.

In one embodiment, the input medical imaging data comprises MRI (magnetic resonance imaging) medical imaging data.

These and other advantages of the invention will be apparent to those of ordinary skill in the art by reference to the following detailed description and the accompanying drawings.

### BRIEF DESCRIPTION OF THE DRAWINGS

FIG. 1 shows a method for determining myocardium strain and intracardiac blood flow of a heart of a patient, in accordance with one or more embodiments;

FIG. 2 shows a workflow for training a cycle GAN (generative adversarial network), in accordance with one or more embodiments;

FIG. 3 shows an exemplary user interface depicting predicted myocardium strain data of the heart and predicted intracardiac blood flow data of the heart overlaid on imaging data of the heart, in accordance with one or more embodiments;

FIG. 4 shows an exemplary artificial neural network that may be used to implement one or more embodiments described herein;

FIG. 5 shows a convolutional neural network that may be used to implement one or more embodiments described herein; and

FIG. 6 shows a high-level block diagram of a computer that may be used to implement one or more embodiments described herein.

### DETAILED DESCRIPTION

The present invention generally relates to methods and systems for the joint assessment of myocardial strain and intracardiac blood flow. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Cardiovascular diseases, such as, e.g., ischemic cardiomyopathy and infraction, non-ischemic cardiomyopathy such as HCM (hypertrophic cardiomyopathy) or amyloidosis, congenital aortic stenosis, and valve diseases, are typically assessed by various types of independently performed MRI examinations. Examples of such MRI examinations include myocardial strain analysis or intracardiac blood flow analysis, which were historically developed independently of each other. Understanding the relationship between myocardial strain and intracardiac blood flow may be beneficial for therapy planning of many cardiovascular diseases. Embodiments described herein provide for a model of the heart of a patient for jointly modelling myocardium strain of the heart and intracardiac blood flow of the heart. In one embodiment, the model of the heart is a computational model of the heart that simulates myocardium strain and intracardiac blood flow of the heart. In another embodiment, the model of the heart is a machine learning based model trained to predict myocardium strain of the heart and intracardiac blood flow of the heart. Advantageously, embodiments described herein enable users to understand the relationship between myocardium strain of the heart and intracardiac blood flow of the heart and their effect on cardiovascular diseases.

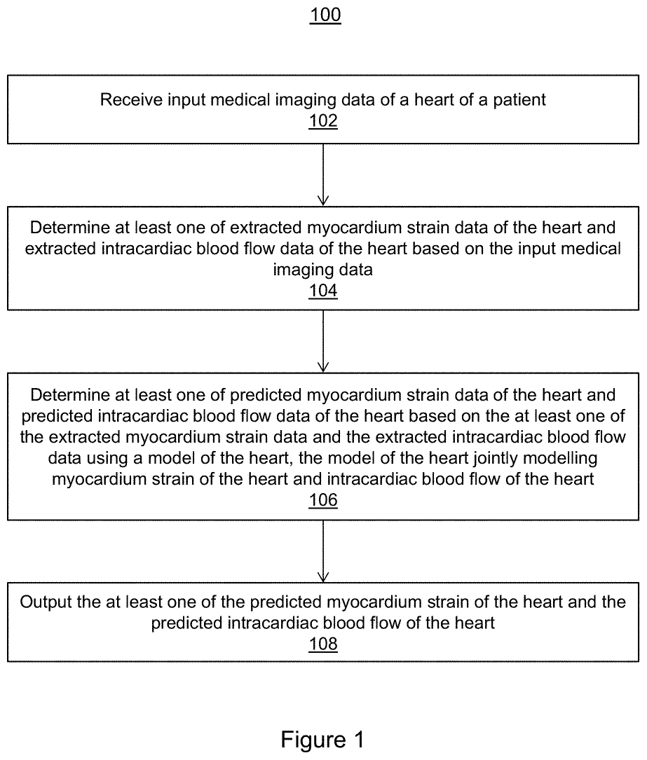

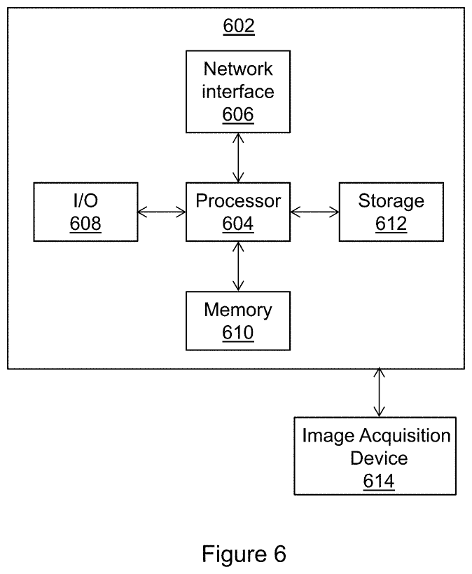

FIG. 1 shows a method 100 for determining myocardium strain and intracardiac blood flow of a heart of a patient, in accordance with one or more embodiments. The steps of method 100 may be performed by one or more suitable computing devices, such as, e.g., computer 602 of FIG. 6.

At step 102, input medical imaging data of a heart of a patient is received. In one embodiment, the input medical imaging data is MRI (magnetic resonance imaging) medical imaging data. For example, the input medical imaging data may be 4D (four dimensional, i.e., three dimensions plus time) MRI medical imaging data comprising a time varying MRI series. However, the input medical imaging data may be of any other suitable modality, such as, e.g., CT (computed tomography), x-ray, US (ultrasound), or any other modality or combination of modalities. The input medical imaging data may comprise 2D (two dimensional) images or 3D (three dimensional) volumes, and may comprise a single image or a plurality of images (e.g., a sequence of images acquired over time). Each pixel or voxel in the input medical imaging data may be associated with a single scalar value, or may be associated with a 3D vector representing a velocity or a deformation. The input medical imaging data may be received directly from an image acquisition device, such as, e.g., an MRI scanner, as the input medical imaging data is acquired, or can be received by loading a previously acquired input medical imaging data from a storage or memory of a computer system or receiving the input medical imaging data from a remote computer system.

At step 104, at least one of extracted myocardium strain data of the heart and extracted intracardiac blood flow data of the heart is determined from the input medical imaging data. The extracted myocardium strain data of the heart may be extracted from the input medical imaging data using known 3D strain imaging techniques, such as, e.g., tagged MRI, DENSE MRI, or tissue phase mapping MRI using dynamic meshes, meshless grid, or particle-based simulations of the myocardium contraction and relaxation with or without using myocardial mass conservation constraints. The extracted intracardiac blood flow data of the heart, representing blood flow in the chambers and optionally the aorta of the heart, may be extracted from the input medical imaging data using known intracardiac blood flow extraction techniques, such as, e.g., direct imaging with 4D flow MRI acquisition methods or CFD (computational fluid dynamics) simulations.

At step 106, at least one of predicted myocardium strain data of the heart and predicted intracardiac blood flow data of the heart is determined based on the at least one of the extracted myocardium strain data and the extracted intracardiac blood flow data using a model of the heart. The model of the heart jointly models myocardium strain of the heart and intracardiac blood flow of the heart.

In one embodiment, the model of the heart is a computational model that simulates the physiological function or operation of the heart. In particular, the computational model of the heart jointly simulates the myocardium strain (the contraction and relaxation of the myocardium) of the heart and the intracardiac blood flow (hemodynamics) of the heart. The computational physiological model of the heart may also simulate other functions or operations, such as, e.g., movement/mechanics of the heart, electrical signal propagation (electrophysiology) of the heart, disease progression of the heart, etc. In one embodiment, the computational model is a fluid structure interaction model implemented using a finite element method, a structured grid method, or a meshless grid or particle-based method. However, the computational model may be any suitable computational model for jointly simulating the myocardium strain of the heart and the intracardiac blood flow of the heart. The computational model of the heart may be generated based on an anatomical model of the heart of the patient extracted from the input medical imaging data. The computational model may be initialized using patient data from a large patient population and personalized by updating parameters of the computational model with the at least one of the extracted myocardium strain data and the extracted intracardiac blood flow data (and possibly other measurements of the patient). The updated computational model may then be used to simulate physiological function of the heart to determine the at least one of the predicted myocardium strain data and the predicted intracardiac blood flow data.

In one embodiment, the model of the heart is a machine learning based model trained to map the extracted myocardium strain data to the predicted intracardiac blood flow data and/or to map the extracted intracardiac blood flow data to the predicted myocardium strain data. The machine learning based model may comprise any suitable machine learning based model, such as, e.g., neural networks, CNNs (convolutional neural networks), deep learning networks, etc. In one embodiment, the machine learning based model is a GAN (generative adversarial network) or a cycle GAN comprising a pair of generator networks jointly trained to respectively map the extracted myocardium strain data to the predicted intracardiac blood flow data and to map the extracted intracardiac blood flow data to the predicted myocardium strain data. The machine learning based model is trained during a prior offline or training stage using a training data set. Once trained, the trained machine learning based model is applied at step 106, during an online or interference stage, to map the extracted myocardium strain data to the predicted intracardiac blood flow data and/or to map the extracted intracardiac blood flow data to the predicted myocardium strain data.

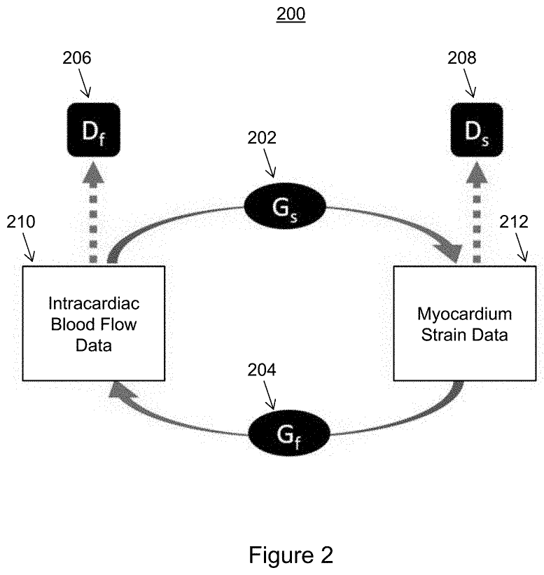

FIG. 2 shows a workflow 200 for training a cycle GAN, in accordance with one or more embodiments. Workflow 200 is performed during a prior offline or training stage for training the cycle GAN. The cycleGAN comprises two generator networks, Generator Gₛ 202 and Generator G_{f} 204, and two discriminator networks, Discriminator Dₛ 208 and Discriminator D_{f} 206. Generator Gₛ 202 generates myocardium strain data 212 from intracardiac blood flow data 210 and Generator G_{f} 204 generates intracardiac blood flow data 210 from myocardium strain data 212. During training, Discriminator Dₛ 208 aims to distinguish between synthesized myocardium strain data 212 (generated by Generator Gₛ 202) and a real myocardium strain data (not shown) and Discriminator D_{f} 206 aims to distinguish between synthesized intracardiac blood flow data 210 (generated by Generator G_{f} 204) and real intracardiac blood flow data (not shown). Accordingly, Generator Gₛ 202 and Generator G_{f} 204 are jointly trained through an adversarial training with Discriminator Dₛ 208 and Discriminator D_{f} 206 to generate realistic myocardium strain data 212 and intracardiac blood flow data 210 respectively. Generator Gₛ 202 and Generator G_{f} 204 of the cycle GAN thereby jointly model myocardium strain of the heart and intracardiac blood flow of the heart. Once trained, the trained cycle GAN is applied during an online or inference stage. In one example, the trained cycle GAN is applied during the online or inference stage to perform step 106 of FIG. 1. During the online or inference stage, only Generator Gₛ 202 and Generator G_{f} 204 are utilized. Discriminator Dₛ 208 and Discriminator D_{f} 206 are only utilized during the training or offline stage.

Returning to FIG. 1, at step 108, the at least one of the predicted myocardium strain data of the heart and the predicted intracardiac blood flow data of the heart is output. For example, the at least one of the predicted myocardium strain data of the heart and the predicted intracardiac blood flow data of the heart can be output by displaying the at least one of the predicted myocardium strain data of the heart and the predicted intracardiac blood flow data of the heart on a display device of a computer system, storing the at least one of the predicted myocardium strain data of the heart and the predicted intracardiac blood flow data of the heart on a memory or storage of a computer system, or by transmitting the at least one of the predicted myocardium strain data of the heart and the predicted intracardiac blood flow data of the heart to a remote computer system.

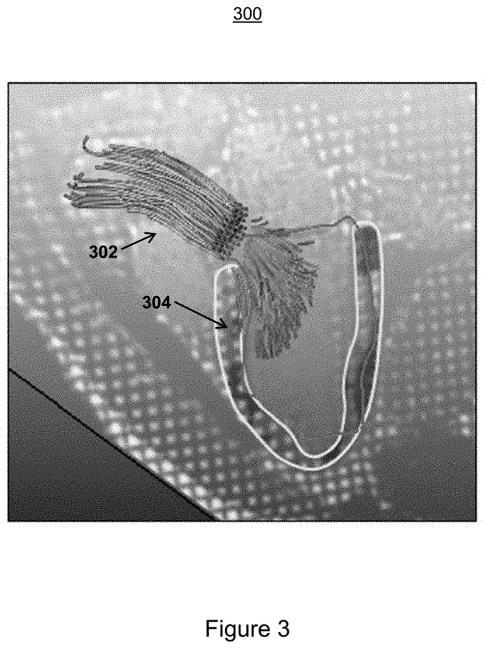

In one embodiment, the predicted myocardium strain data of the heart and the predicted intracardiac blood flow data of the heart are output by displaying both the predicted myocardium strain data of the heart and the predicted intracardiac blood flow data of the heart overlaid on the input medical imaging data. FIG. 3 shows an exemplary user interface 300 depicting predicted myocardium strain data of the heart and predicted intracardiac blood flow data of the heart overlaid on a 2D image data that represents a cross-section of the heart, in accordance with one or more embodiments. User interface 300 depicts predicted myocardium strain data 302 and predicted intracardiac blood flow data 304. Predicted myocardium strain data 302 may be depicted as local strain visualizations, such as, e.g., color maps of the magnitude of various scalar strain measures or displacement vectors, which may be visualized on 2D planes (e.g., cross sections of the endocardium or AHA (American Heart Association) segments), surfaces (e.g., meshes of the endo, epi, or mid-myocardium boundaries), volumes (e.g., volumetric rendering of the heart), or other graphical representations of the heart of the patient, which may be generated based on the input medical imaging data. Predicted intracardiac blood flow data 304 may be depicted as flow visualizations, such as, e.g., velocity vectors, particle traces, stream lines, path lines, or streak lines. These visualizations can be dynamic (change over time for each timepoint in the data), or may integrate the dynamic information in one static 2D or 3D rendering. In one embodiment, user interface 300 may include a custom lens tool to enable a user to interactively inspect predicted myocardium strain data 302 and predicted intracardiac blood flow data 304 in user selected segments of the myocardium.

In one embodiment, the computational model of the heart may be utilized to correct the extracted myocardium strain data and the extracted intracardiac blood flow data. Typically, blood flow closer to vessel walls or in the atrial appendage is slower. However, the lower velocity blood flow may not be accurately reflected in the measured intracardiac blood flow data. In one embodiment, to increase the fidelity of the blood flow, the computational model may be constrained by the extracted myocardium strain data and the extracted intracardiac blood flow data. The constrained computational model is used to simulate intracardiac blood flow using computational fluid dynamics, taking account cardiac chamber contractility or deformation in the form of strain parameters. Accordingly, the constrained computational model may determine the predicted intracardiac blood flow data as the blood flow that most closely satisfies both the extracted myocardium strain data and the extracted intracardiac blood flow data. The extracted intracardiac blood flow may be corrected to be consistent with the predicted intracardiac blood flow determined by the constrained computational model. Similarly, the constrained computational model may be utilized to correct the extracted myocardium strain data to be consistent with the predicted myocardium strain data. In some embodiments, strain or flow measurements from an echocardiogram may be applied to constrain the computational model.

In one embodiment, the computational model may be utilized to simulate disease progression. The simulated disease progression may be used as a learning tool or to inform patients of possible long term implications of unhealthy behavior or implications of failing to follow advised treatments and diet. The simulated disease progression may provide time lapse scenarios for disease progression where the disease is untreated, given adverse symptoms such as, e.g., abnormal pressure gradient, reduced contractility, or myocardial wall morphological anomalies to illustrate possible conditions under which symptoms may worsen and lead to heart failure. The simulated disease progression may later be validated through longitudinal studies.

In one embodiment, the computational model may be utilized to simulate the most common cardiovascular diseases to obtain myocardial strain changes based on projected blood flow parameter changes, and vice versa. For example, myocardial wall thickness and contractility and recovery dynamics may be modified in the computational model to simulate HCM progression or possible effects of medication to enable a clinician (or other user) to observe if such modifications result in changes to intracardiac flow patterns and relative pressure. In another example, to simulate valve disease progression, the computational model may be updated to model the desired correction in pressure gradient that could be achieved through invasive valve replacement. The constrained computational model may then simulate changes in the myocardial contraction pattern to model, for example, how contraction patterns are progressively affected by changes in valve regurgitant fraction or if a medication induced decrease in contraction could prevent valve reopening or decrease regurgitation.

Embodiments described herein are described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the providing system.

Furthermore, certain embodiments described herein are described with respect to methods and systems for the joint assessment of myocardial strain and intracardial blood flow using machine learning based networks (or models), as well as with respect to methods and systems for training a machine learning based network for the joint assessment of myocardial strain and intracardial blood flow. Features, advantages or alternative embodiments herein can be assigned to the other claimed objects and vice versa. In other words, claims for methods and systems for training a machine learning based network can be improved with features described or claimed in context of the methods and systems for utilizing a trained machine learning based network, and vice versa.

In particular, the trained machine learning based networks applied in the methods and systems for the joint assessment of myocardial strain and intracardial blood flow can be adapted by the methods and systems for training the machine learning based network for the joint assessment of myocardial strain and intracardial blood flow. Furthermore, the input data of the trained machine learning based segmentation network can comprise advantageous features and embodiments of the training input data, and vice versa. Furthermore, the output data of the trained machine learning based network can comprise advantageous features and embodiments of the output training data, and vice versa.

In general, a trained machine learning based network mimics cognitive functions that humans associate with other human minds. In particular, by training based on training data, the trained machine learning based network is able to adapt to new circumstances and to detect and extrapolate patterns.

In general, parameters of a machine learning based network can be adapted by means of training. In particular, supervised training, semi-supervised training, unsupervised training, reinforcement learning and/or active learning can be used. Furthermore, representation learning (an alternative term is “feature learning”) can be used. In particular, the parameters of the trained machine learning based network can be adapted iteratively by several steps of training.

In particular, a trained machine learning based network can comprise a neural network, a support vector machine, a decision tree, and/or a Bayesian network, and/or the trained machine learning based network can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network and/or a generative adversarial network.

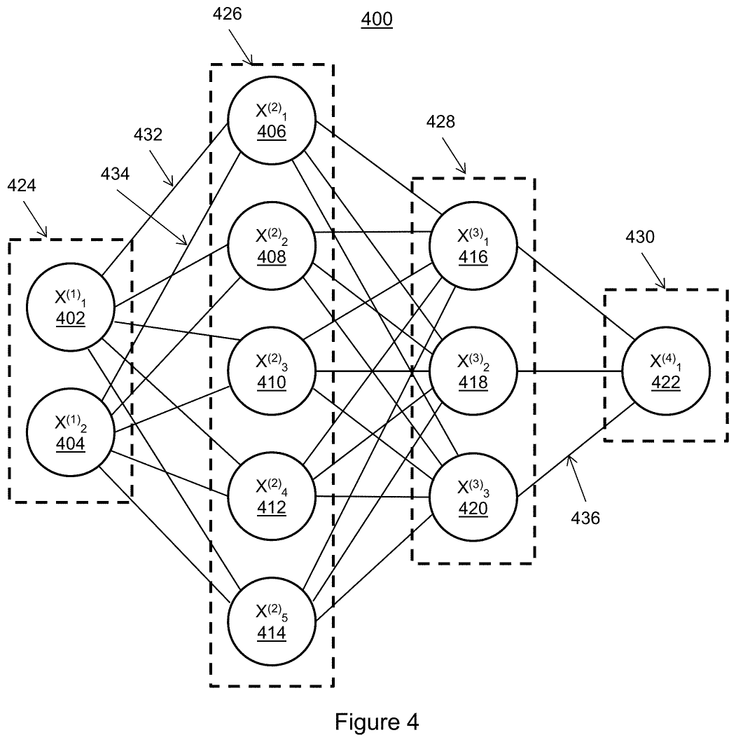

FIG. 4 shows an embodiment of an artificial neural network 400, in accordance with one or more embodiments. Alternative terms for “artificial neural network” are “neural network”, “artificial neural net” or “neural net”. Machine learning networks described herein, such as, e.g., the machine learning based model applied at step 106 of FIG. 1 or the cycle GAN shown in FIG. 2, may be implemented using artificial neural network 400.

The artificial neural network 400 comprises nodes 402-422 and edges 432, 434, . . . , 436, wherein each edge 432, 434, . . . , 436 is a directed connection from a first node 402-422 to a second node 402-422. In general, the first node 402-422 and the second node 402-422 are different nodes 402-422, it is also possible that the first node 402-422 and the second node 402-422 are identical. For example, in FIG. 4, the edge 432 is a directed connection from the node 402 to the node 406, and the edge 434 is a directed connection from the node 404 to the node 406. An edge 432, 434, . . . , 436 from a first node 402-422 to a second node 402-422 is also denoted as “ingoing edge” for the second node 402-422 and as “outgoing edge” for the first node 402-422.

In this embodiment, the nodes 402-422 of the artificial neural network 400 can be arranged in layers 424-430, wherein the layers can comprise an intrinsic order introduced by the edges 432, 434, . . . , 436 between the nodes 402-422. In particular, edges 432, 434, . . . , 436 can exist only between neighboring layers of nodes. In the embodiment shown in FIG. 4, there is an input layer 424 comprising only nodes 402 and 404 without an incoming edge, an output layer 430 comprising only node 422 without outgoing edges, and hidden layers 426, 428 in-between the input layer 424 and the output layer 430. In general, the number of hidden layers 426, 428 can be chosen arbitrarily. The number of nodes 402 and 404 within the input layer 424 usually relates to the number of input values of the neural network 400, and the number of nodes 422 within the output layer 430 usually relates to the number of output values of the neural network 400.

In particular, a (real) number can be assigned as a value to every node 402-422 of the neural network 400. Here, x⁽ⁿ⁾ᵢ denotes the value of the i-th node 402-422 of the n-th layer 424-430. The values of the nodes 402-422 of the input layer 424 are equivalent to the input values of the neural network 400, the value of the node 422 of the output layer 430 is equivalent to the output value of the neural network 400. Furthermore, each edge 432, 434, . . . , 436 can comprise a weight being a real number, in particular, the weight is a real number within the interval [−1, 1] or within the interval [0, 1]. Here, w^{(m,n)}_{i,j} denotes the weight of the edge between the i-th node 402-422 of the m-th layer 424-430 and the j-th node 402-422 of the n-th layer 424-430. Furthermore, the abbreviation w⁽ⁿ⁾_{i,j} is defined for the weight w^{(n,n+1)}_{i,j}.

In particular, to calculate the output values of the neural network 400, the input values are propagated through the neural network. In particular, the values of the nodes 402-422 of the (n+1)-th layer 424-430 can be calculated based on the values of the nodes 402-422 of the n-th layer 424-430 by

xⱼ⁽ⁿ⁺¹⁾=f(Σᵢxᵢ⁽ⁿ⁾·w_{i,j}⁽ⁿ⁾).

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 424 are given by the input of the neural network 400, wherein values of the first hidden layer 426 can be calculated based on the values of the input layer 424 of the neural network, wherein values of the second hidden layer 428 can be calculated based in the values of the first hidden layer 426, etc.

In order to set the values w^{(m,n)}_{i,j} for the edges, the neural network 400 has to be trained using training data. In particular, training data comprises training input data and training output data (denoted as tᵢ). For a training step, the neural network 400 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 400 (backpropagation algorithm). In particular, the weights are changed according to

w_{i,j}¹⁽ⁿ⁾=w_{i,j}⁽ⁿ⁾−γ·δⱼ⁽ⁿ⁾·xᵢ⁽ⁿ⁾

wherein γ is a learning rate, and the numbers δ⁽ⁿ⁾ⱼ can be recursively calculated as

δⱼ⁽ⁿ⁾=(Σₖδₖ⁽ⁿ⁺¹⁾·w_{j,k}⁽ⁿ⁺¹⁾)·f′(Σᵢxᵢ⁽ⁿ⁾·w_{i,j}⁽ⁿ⁾)

based on δ⁽ⁿ⁺¹⁾ⱼ, if the (n+1)-th layer is not the output layer, and

δⱼ⁽ⁿ⁾=(xₖ⁽ⁿ⁺¹⁾−tⱼ⁽ⁿ⁺¹⁾)·f′(Σᵢxᵢ⁽ⁿ⁾·w_{i,j}⁽ⁿ⁾)

if the (n+1)-th layer is the output layer 430, wherein f′ is the first derivative of the activation function, and y⁽ⁿ⁺¹⁾ⱼ is the comparison training value for the j-th node of the output layer 430.

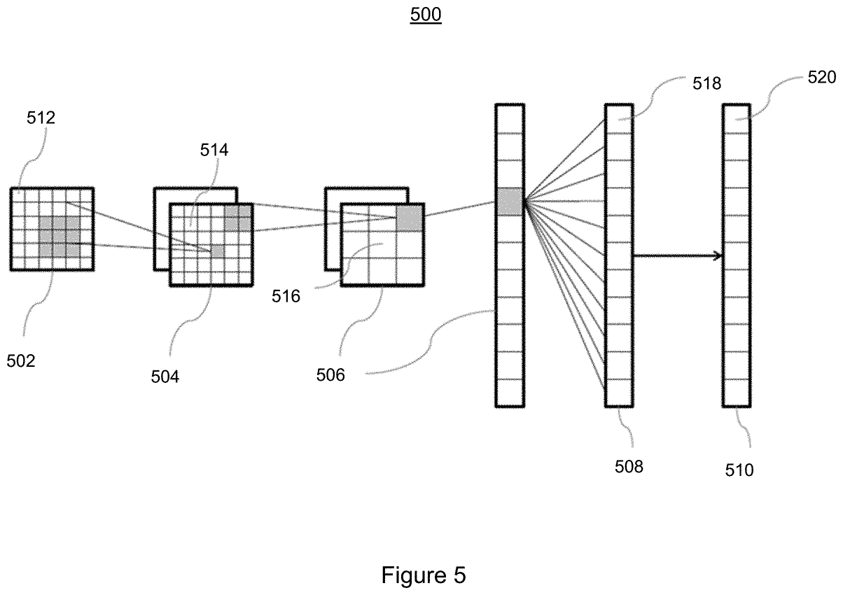

FIG. 5 shows a convolutional neural network 500, in accordance with one or more embodiments. Machine learning networks described herein, such as, e.g., the machine learning based model applied at step 106 of FIG. 1 or the cycle GAN shown in FIG. 2, may be implemented using convolutional neural network 500.

In the embodiment shown in FIG. 5, the convolutional neural network comprises 500 an input layer 502, a convolutional layer 504, a pooling layer 506, a fully connected layer 508, and an output layer 510. Alternatively, the convolutional neural network 500 can comprise several convolutional layers 504, several pooling layers 506, and several fully connected layers 508, as well as other types of layers. The order of the layers can be chosen arbitrarily, usually fully connected layers 508 are used as the last layers before the output layer 510.

In particular, within a convolutional neural network 500, the nodes 512-520 of one layer 502-510 can be considered to be arranged as a d-dimensional matrix or as a d-dimensional image. In particular, in the two-dimensional case the value of the node 512-520 indexed with i and j in the n-th layer 502-510 can be denoted as x⁽ⁿ_{)[i,j]}. However, the arrangement of the nodes 512-520 of one layer 502-510 does not have an effect on the calculations executed within the convolutional neural network 500 as such, since these are given solely by the structure and the weights of the edges.

In particular, a convolutional layer 504 is characterized by the structure and the weights of the incoming edges forming a convolution operation based on a certain number of kernels. In particular, the structure and the weights of the incoming edges are chosen such that the values x⁽ⁿ⁾ₖ of the nodes 514 of the convolutional layer 504 are calculated as a convolution x⁽ⁿ⁾ₖ=Kₖ*x⁽ⁿ⁻¹⁾ based on the values x⁽ⁿ⁻¹⁾ of the nodes 512 of the preceding layer 502, where the convolution * is defined in the two-dimensional case as

xₖ⁽ⁿ⁾[i,j]=(Kₖ*x⁽ⁿ⁻¹⁾)[i,j]=Σ_{i′}Σ_{j′}Kₖ[i′,j′]·x⁽ⁿ⁻¹⁾[i-i′,j-j′].

Here the k-th kernel Kₖ is a d-dimensional matrix (in this embodiment a two-dimensional matrix), which is usually small compared to the number of nodes 512-518 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 512-520 in the respective layer 502-510. In particular, for a convolutional layer 504, the number of nodes 514 in the convolutional layer is equivalent to the number of nodes 512 in the preceding layer 502 multiplied with the number of kernels.

If the nodes 512 of the preceding layer 502 are arranged as a d-dimensional matrix, using a plurality of kernels can be interpreted as adding a further dimension (denoted as “depth” dimension), so that the nodes 514 of the convolutional layer 504 are arranged as a (d+1)-dimensional matrix. If the nodes 512 of the preceding layer 502 are already arranged as a (d+1)-dimensional matrix comprising a depth dimension, using a plurality of kernels can be interpreted as expanding along the depth dimension, so that the nodes 514 of the convolutional layer 504 are arranged also as a (d+1)-dimensional matrix, wherein the size of the (d+1)-dimensional matrix with respect to the depth dimension is by a factor of the number of kernels larger than in the preceding layer 502.

The advantage of using convolutional layers 504 is that spatially local correlation of the input data can exploited by enforcing a local connectivity pattern between nodes of adjacent layers, in particular by each node being connected to only a small region of the nodes of the preceding layer.

In embodiment shown in FIG. 5, the input layer 502 comprises 36 nodes 512, arranged as a two-dimensional 6×6 matrix. The convolutional layer 504 comprises 72 nodes 514, arranged as two two-dimensional 6×6 matrices, each of the two matrices being the result of a convolution of the values of the input layer with a kernel. Equivalently, the nodes 514 of the convolutional layer 504 can be interpreted as arranges as a three-dimensional 6×6×2 matrix, wherein the last dimension is the depth dimension.

A pooling layer 506 can be characterized by the structure and the weights of the incoming edges and the activation function of its nodes 516 forming a pooling operation based on a non-linear pooling function f. For example, in the two dimensional case the values x⁽ⁿ⁾ of the nodes 516 of the pooling layer 506 can be calculated based on the values x⁽ⁿ⁻¹⁾ of the nodes 514 of the preceding layer 504 as

x⁽ⁿ⁾[i,j]=f(x⁽ⁿ⁻¹⁾[id₁,jd₂], . . . x⁽ⁿ⁻¹⁾[id₁+d₁−1,jd₂+d₂−1])

In other words, by using a pooling layer 506, the number of nodes 514, 516 can be reduced, by replacing a number d1·d2 of neighboring nodes 514 in the preceding layer 504 with a single node 516 being calculated as a function of the values of said number of neighboring nodes in the pooling layer. In particular, the pooling function f can be the max-function, the average or the L2-Norm. In particular, for a pooling layer 506 the weights of the incoming edges are fixed and are not modified by training.

The advantage of using a pooling layer 506 is that the number of nodes 514, 516 and the number of parameters is reduced. This leads to the amount of computation in the network being reduced and to a control of overfitting.

In the embodiment shown in FIG. 5, the pooling layer 506 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 508 can be characterized by the fact that a majority, in particular, all edges between nodes 516 of the previous layer 506 and the nodes 518 of the fully-connected layer 508 are present, and wherein the weight of each of the edges can be adjusted individually.

In this embodiment, the nodes 516 of the preceding layer 506 of the fully-connected layer 508 are displayed both as two-dimensional matrices, and additionally as non-related nodes (indicated as a line of nodes, wherein the number of nodes was reduced for a better presentability). In this embodiment, the number of nodes 518 in the fully connected layer 508 is equal to the number of nodes 516 in the preceding layer 506. Alternatively, the number of nodes 516, 518 can differ.

Furthermore, in this embodiment, the values of the nodes 520 of the output layer 510 are determined by applying the Softmax function onto the values of the nodes 518 of the preceding layer 508. By applying the Softmax function, the sum the values of all nodes 520 of the output layer 510 is 1, and all values of all nodes 520 of the output layer are real numbers between 0 and 1.

A convolutional neural network 500 can also comprise a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks can be wired using summation (residual/dense neural networks), element-wise multiplication (attention) or other differentiable operators. Therefore, the convolutional neural network architecture can be nested rather than being sequential if the whole pipeline is differentiable.

In particular, convolutional neural networks 500 can be trained based on the backpropagation algorithm. For preventing overfitting, methods of regularization can be used, e.g. dropout of nodes 512-520, stochastic pooling, use of artificial data, weight decay based on the L1 or the L2 norm, or max norm constraints. Different loss functions can be combined for training the same neural network to reflect the joint training objectives. A subset of the neural network parameters can be excluded from optimization to retain the weights pretrained on another datasets.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1. Certain steps or functions of the methods and workflows described herein, including one or more of the steps or functions of FIG. 1, may be performed by a server or by another processor in a network-based cloud-computing system. Certain steps or functions of the methods and workflows described herein, including one or more of the steps of FIG. 1, may be performed by a client computer in a network-based cloud computing system. The steps or functions of the methods and workflows described herein, including one or more of the steps of FIG. 1, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 602 that may be used to implement systems, apparatus, and methods described herein is depicted in FIG. 6. Computer 602 includes a processor 604 operatively coupled to a data storage device 612 and a memory 610. Processor 604 controls the overall operation of computer 602 by executing computer program instructions that define such operations. The computer program instructions may be stored in data storage device 612, or other computer readable medium, and loaded into memory 610 when execution of the computer program instructions is desired. Thus, the method and workflow steps or functions of FIGS. 2 and 4 can be defined by the computer program instructions stored in memory 610 and/or data storage device 612 and controlled by processor 604 executing the computer program instructions. For example, the computer program instructions can be implemented as computer executable code programmed by one skilled in the art to perform the method and workflow steps or functions of FIG. 1. Accordingly, by executing the computer program instructions, the processor 604 executes the method and workflow steps or functions of FIG. 1. Computer 602 may also include one or more network interfaces 606 for communicating with other devices via a network. Computer 602 may also include one or more input/output devices 608 that enable user interaction with computer 602 (e.g., display, keyboard, mouse, speakers, buttons, etc.).

Processor 604 may include both general and special purpose microprocessors, and may be the sole processor or one of multiple processors of computer 602. Processor 604 may include one or more central processing units (CPUs), for example. Processor 604, data storage device 612, and/or memory 610 may include, be supplemented by, or incorporated in, one or more application-specific integrated circuits (ASICs) and/or one or more field programmable gate arrays (FPGAs).

Data storage device 612 and memory 610 each include a tangible non-transitory computer readable storage medium. Data storage device 612, and memory 610, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 608 may include peripherals, such as a printer, scanner, display screen, etc. For example, input/output devices 608 may include a display device such as a cathode ray tube (CRT) or liquid crystal display (LCD) monitor for displaying information to the user, a keyboard, and a pointing device such as a mouse or a trackball by which the user can provide input to computer 602.

An image acquisition device 614 can be connected to the computer 602 to input image data (e.g., medical images) to the computer 602. It is possible to implement the image acquisition device 614 and the computer 602 as one device. It is also possible that the image acquisition device 614 and the computer 602 communicate wirelessly through a network. In a possible embodiment, the computer 602 can be located remotely with respect to the image acquisition device 614.

Any or all of the systems and apparatus discussed herein, the machine learning based model applied at step 106 of FIG. 1 or the cycle GAN shown in FIG. 2, may be implemented using one or more computers such as computer 602.

One skilled in the art will recognize that an implementation of an actual computer or computer system may have other structures and may contain other components as well, and that FIG. 6 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method comprising:
receiving input medical imaging data of a heart of a patient;
determining at least one of extracted myocardium strain data of the heart and extracted intracardiac blood flow data of the heart from the input medical imaging data;
determining at least one of predicted myocardium strain data of the heart and predicted intracardiac blood flow data of the heart based on the at least one of the extracted myocardium strain data and the extracted intracardiac blood flow data using a model of the heart, the model of the heart jointly modelling myocardium strain of the heart and intracardiac blood flow of the heart; and
outputting the at least one of the predicted myocardium strain of the heart and the predicted intracardiac blood flow of the heart.

2. The method of claim 1, wherein the model of the heart is a computational model of the heart, and determining at least one of predicted myocardium strain data of the heart and predicted intracardiac blood flow data of the heart based on the at least one of the extracted myocardium strain data and the extracted intracardiac blood flow data using a model of the heart comprises:
updating the computational model of the heart based on the extracted myocardium strain data of the heart and extracted intracardiac blood flow data; and
simulating physiological function of the heart using the updated computational model to determine the at least one of the predicted myocardium strain data of the heart and the predicted intracardiac blood flow data of the heart.

3. The method of claim 1, wherein the model of the heart is a machine learning based model of the heart, and determining at least one of predicted myocardium strain data of the heart and predicted intracardiac blood flow data of the heart based on the at least one of the extracted myocardium strain data and the extracted intracardiac blood flow data using a model of the heart comprises:
predicting the predicted myocardium strain data of the heart from the extracted intracardiac blood flow data of the heart using the machine learning based model; and
predicting the predicted intracardiac blood flow data of the heart from the extracted myocardium strain data of the heart using the machine learning based model.

4. The method of claim 3, wherein the machine learning based model of the heart comprises a first generator network for predicting the predicted myocardium strain data of the heart from the extracted intracardiac blood flow data of the heart and a second generator network for predicting the predicted intracardiac blood flow data of the heart from the extracted myocardium strain data of the heart.

5. The method of claim 4, wherein the first generator network and the second generator network are jointly trained through an adversarial training with a first discriminator network and a second discriminator network, respectively.

6. The method of claim 1, wherein outputting the at least one of the predicted myocardium strain of the heart and the predicted intracardiac blood flow of the heart comprises:
displaying the at least one of the predicted myocardium strain of the heart and the predicted intracardiac blood flow of the heart overlaid on the input medical imaging data.

7. The method of claim 1, wherein outputting the at least one of the predicted myocardium strain of the heart and the predicted intracardiac blood flow of the heart comprises:
displaying the at least one of the predicted myocardium strain of the heart and the predicted intracardiac blood flow of the heart overlaid on a graphical representation of the heart of the patient generated based on the input medical imaging data.

8. The method of claim 1, further comprising:
simulating progression of a disease using the model of the heart.

9. The method of claim 1, wherein the input medical imaging data comprises MRI (magnetic resonance imaging) medical imaging data.

10. An apparatus comprising:
means for receiving input medical imaging data of a heart of a patient;
means for determining at least one of extracted myocardium strain data of the heart and extracted intracardiac blood flow data of the heart from the input medical imaging data;
means for determining at least one of predicted myocardium strain data of the heart and predicted intracardiac blood flow data of the heart based on the at least one of the extracted myocardium strain data and the extracted intracardiac blood flow data using a model of the heart, the model of the heart jointly modelling myocardium strain of the heart and intracardiac blood flow of the heart; and
means for outputting the at least one of the predicted myocardium strain of the heart and the predicted intracardiac blood flow of the heart.

11. The apparatus of claim 10, wherein the model of the heart is a computational model of the heart, and the means for determining at least one of predicted myocardium strain data of the heart and predicted intracardiac blood flow data of the heart based on the at least one of the extracted myocardium strain data and the extracted intracardiac blood flow data using a model of the heart comprises:
means for updating the computational model of the heart based on the extracted myocardium strain data of the heart and extracted intracardiac blood flow data; and
means for simulating physiological function of the heart using the updated computational model to determine the at least one of the predicted myocardium strain data of the heart and the predicted intracardiac blood flow data of the heart.

12. The apparatus of claim 10, wherein the model of the heart is a machine learning based model of the heart, and the means for determining at least one of predicted myocardium strain data of the heart and predicted intracardiac blood flow data of the heart based on the at least one of the extracted myocardium strain data and the extracted intracardiac blood flow data using a model of the heart comprises:
means for predicting the predicted myocardium strain data of the heart from the extracted intracardiac blood flow data of the heart using the machine learning based model; and
means for predicting the predicted intracardiac blood flow data of the heart from the extracted myocardium strain data of the heart using the machine learning based model.

13. The apparatus of claim 12, wherein the machine learning based model of the heart comprises a first generator network for predicting the predicted myocardium strain data of the heart from the extracted intracardiac blood flow data of the heart and a second generator network for predicting the predicted intracardiac blood flow data of the heart from the extracted myocardium strain data of the heart.

14. The apparatus of claim 13, wherein the first generator network and the second generator network are jointly trained through an adversarial training with a first discriminator network and a second discriminator network, respectively.

15. A non-transitory computer readable medium storing computer program instructions, the computer program instructions when executed by a processor cause the processor to perform operations comprising:
receiving input medical imaging data of a heart of a patient;
determining at least one of extracted myocardium strain data of the heart and extracted intracardiac blood flow data of the heart from the input medical imaging data;
determining at least one of predicted myocardium strain data of the heart and predicted intracardiac blood flow data of the heart based on the at least one of the extracted myocardium strain data and the extracted intracardiac blood flow data using a model of the heart, the model of the heart jointly modelling myocardium strain of the heart and intracardiac blood flow of the heart; and
outputting the at least one of the predicted myocardium strain of the heart and the predicted intracardiac blood flow of the heart.

16. The non-transitory computer readable medium of claim 15, wherein the model of the heart is a computational model of the heart, and determining at least one of predicted myocardium strain data of the heart and predicted intracardiac blood flow data of the heart based on the at least one of the extracted myocardium strain data and the extracted intracardiac blood flow data using a model of the heart comprises:
updating the computational model of the heart based on the extracted myocardium strain data of the heart and extracted intracardiac blood flow data; and
simulating physiological function of the heart using the updated computational model to determine the at least one of the predicted myocardium strain data of the heart and the predicted intracardiac blood flow data of the heart.

17. The non-transitory computer readable medium of claim 15, wherein outputting the at least one of the predicted myocardium strain of the heart and the predicted intracardiac blood flow of the heart comprises:
displaying the at least one of the predicted myocardium strain of the heart and the predicted intracardiac blood flow of the heart overlaid on the input medical imaging data.

18. The non-transitory computer readable medium of claim 15, wherein outputting the at least one of the predicted myocardium strain of the heart and the predicted intracardiac blood flow of the heart comprises:
displaying the at least one of the predicted myocardium strain of the heart and the predicted intracardiac blood flow of the heart overlaid on a graphical representation of the heart of the patient generated based on the input medical imaging data.

19. The non-transitory computer readable medium of claim 15, the operations further comprising:
simulating progression of a disease using the model of the heart.

20. The non-transitory computer readable medium of claim 15, wherein the input medical imaging data comprises MRI (magnetic resonance imaging) medical imaging data.
